(19) United States
(12) Patent Application Publication
(10) Pub. No.: US 2015/0103973 A1
(43) Pub. Date: Apr. 16, 2015

(54) X-RAY SYSTEM WITH MULTIPLE DYNAMIC RANGE SELECTIONS

(71) Applicant: General Electric Company, Schenectady, NY (US)

(72) Inventors: James Zhengshe Liu, Salt Lake City, UT (US); Jonathan Edvard Snyder, Salt Lake City, UT (US); Richard Larry Anderton, Salt Lake City, UT (US); David Ellis Barker, Salt Lake City, UT (US); Brian James Grekowicz, Salt Lake City, UT (US)

(73) Assignee: General Electric Company, Schenectady, NY (US)

(21) Appl. No.: 14/052,026

(22) Filed: Oct. 11, 2013

(51) Int. Cl. G01T 1/208; G01T 1/20; G01N 23/223; G01N 23/02; G01N 23/04

(52) U.S. Cl. CPC G01T 1/208; G01N 23/02; G01N 23/04; G01N 23/223; G01T 1/2018
USPC 378/44; 378/51; 378/62

(57) ABSTRACT

An x-ray system with multiple dynamic range selections. The system including an x-ray source and a detector, wherein the detector includes a scintillator and a pixel. The pixel includes a photodiode and first and second capacitors connectable to the photodiode. The pixel is configured to be switched between a first state where only the first capacitor is connected to the photodiode and a second state where both capacitors are connected to the photodiode. The x-ray source directs x-rays to the detector and a region of interest positioned between the source and detector, the scintillator converts the x-rays to light, and the pixel photodiode converts the light to an electrical charge. The charge is stored in the first capacitor when the pixel is in the first state and in the first and second capacitors when the pixel is in the second state.

### BACKGROUND

Digital x-ray systems, such as C-arm x-ray systems or fixed room x-ray systems, are used by healthcare professionals to acquire images of a region of interest (“ROI”) of a patient. These systems include an x-ray source and an x-ray detector that includes a digital image capture device. The ROI is positioned between the x-ray source and x-ray detector and a dose of x-rays is directed at the ROI. Based on the x-ray energy that passes through the ROI to the x-ray detector, a digital x-ray image is made of the ROI. This x-ray image typically includes variations in the brightness of the image or what is known as image “noise.”

The noise in an x-ray image comes from a combination of quantum noise, electronic noise, and quantization noise. Quantum noise is proportional to the number of x-ray photons that arrive at the x-ray detector. Thus, quantum noise increases with the x-ray dose for a procedure. Electronic noise is related to the thermal noise of the x-ray detector and is independent of the x-ray dose. Electronic noise is a function of the maximum electrical charge that the x-ray detector can hold, i.e., the dynamic range of the x-ray system. The larger the dynamic range of the x-ray detector, the greater the electronic noise. Quantization noise occurs when the analog signal of the electrons received by the x-ray detector are converted into a digital signal. Quantization noise is typically very low compared to electronic and quantum noise.

Thus, image noise can be reduced by reducing the dynamic range of the detector (and thus reducing the electronic noise). However, for certain applications, an x-ray system needs to be able to apply a large x-ray dose and include a detector with a large dynamic range.

### SUMMARY OF EMBODIMENTS OF THE INVENTION

Certain embodiments of the present invention provide an x-ray system that includes an x-ray source and an x-ray detector. The x-ray detector includes a scintillator and at least one pixel. The pixel includes a photodiode, a first capacitor connectable to the photodiode, and a second capacitor connectable to the photodiode. The at least one pixel is configured to be switched between a first state wherein only the first capacitor is operatively connected to the photodiode and a second state wherein the first and second capacitors are both operatively connected to the photodiode. The x-ray source directs x-rays at a region of interest positioned between the x-ray source and the x-ray detector, and the scintillator converts the x-rays to light. The photodiode of the at least one pixel converts the light to an electrical charge, and the charge is stored in the first capacitor when the at least one pixel is in the first state and the charge is stored in the first and second capacitors when the at least one pixel is in the second state.

The at least one pixel may also include a third capacitor connectable to the photodiode. The pixel may be configured to be switched from the first or second state to a third state wherein the first, second, and third capacitors are all operatively connected to the photodiode

Certain embodiments of the present invention provide a method for changing the maximum electrical charge that an x-ray detector can hold. The method includes the steps of providing an x-ray system having an x-ray source and an x-ray detector connected to a controller, wherein the x-ray detector includes a scintillator and a pixel including a photodiode connectable to a first capacitor and a second capacitor. The method further includes the steps of selecting, with the controller, to connect only the first capacitor to the photodiode or to connect both the first capacitor and the second capacitor to the photodiode, directing an x-ray dose from the x-ray source at a region of interest positioned between the x-ray source and x-ray detector, and converting the x-rays of the x-ray dose to light with the scintillator. The method further includes converting the light into an electrical charge with the photodiode of the pixel and storing the charge in the first capacitor if only the first capacitor was selected to be connected to the photodiode or in the first and second capacitors if both the first and second capacitors were selected to be connected to the photodiode. The method includes the steps of converting the charge into a digital signal and converting the digital signal to an image of the region of interest.

The method may also include providing a third capacitor that is connectable to the photodiode of the pixel and selecting to connect only the first capacitor to the photodiode, or to connect only the first and second capacitors to the photodiode, or to connect the first, second, and third capacitors to the photodiode.

Certain embodiments of the present invention provide a method for changing the maximum electrical charge that an x-ray detector can hold. The method includes the steps of providing an x-ray system configured to create an x-ray image of a region of interest and having an x-ray source and an x-ray detector connected to a controller, wherein the x-ray detector includes at least one pixel including a photodiode connectable to a first capacitor and at least a second capacitor. The method also includes the steps of connecting only the first capacitor to the photodiode and leaving the at least a second capacitor unconnected to the photodiode, positioning a region of interest between the x-ray source and x-ray detector, and directing x-rays at the region of interest to acquire an image of the region of interest. The method also includes the steps of calculating the average count of electrons received by the detector from the step of directing x-rays at multiple small regions of interest within the region of interest and comparing the maximal average count of electrons among the small regions of interest to a threshold count of electrons that corresponds to the number of electrons that can be held by the at least one pixel when the photodiode of the at least one pixel is connected to the first capacitor.

The method may also include selecting to connect the at least a second capacitor to the photodiode of the pixel if the maximal average count is greater than the threshold count.

The method may further include adjusting the gain associated with an image taken with the first and at least second capacitor connected to the photodiode such that brightness of the displayed image looks the same or similar to that of the image taken with only the first capacitor connected to the photodiode.

### BRIEF DESCRIPTION OF SEVERAL VIEWS OF THE DRAWINGS

FIG. 1 illustrates a block diagram of an embodiment of a digital X-ray imaging system for acquiring original image data and processing the image data for display.

FIG. 2 illustrates a cutaway side view of a portion of an image detector formed in accordance with an embodiment of the present invention.

FIG. 3 illustrates a diagram of a pixel circuit formed in accordance with an embodiment of the present invention.

FIG. 4 illustrates a flow diagram for selecting a dynamic range for an x-ray imaging procedure formed in accordance with an embodiment of the present invention.

FIG. 5 illustrates a flow diagram for selecting a dynamic range and adjusting image brightness for an x-ray imaging procedure formed in accordance with an embodiment of the present invention

Before the embodiments of the invention are explained in detail, it is to be understood that the invention is not limited in its application to the details of construction and the arrangement of the components set forth in the following description or illustrated in the drawings. The invention is capable of other embodiments and of being practiced or being carried out in various ways. Also, it is to be understood that the phraseology and terminology used herein are for the purpose of description and should not be regarded as limiting. The use of “including” and “comprising” and variations thereof is meant to encompass the items listed thereafter and equivalents thereof as well as additional items and equivalents thereof.

### DETAILED DESCRIPTION OF EMBODIMENTS OF THE INVENTION

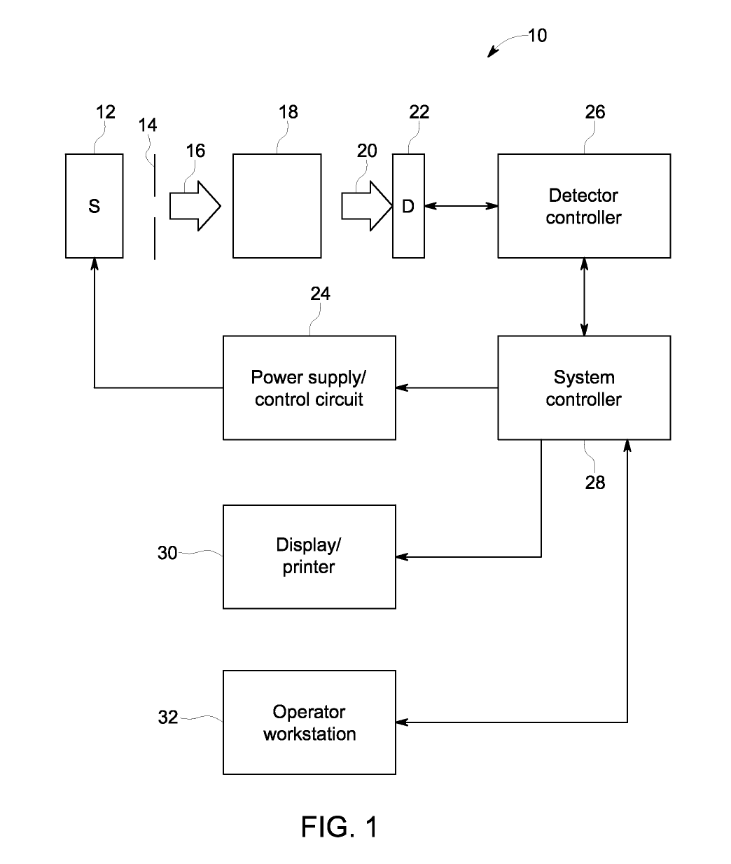

Referring to the drawings, FIG. 1 illustrates a block diagram of an exemplary embodiment of a digital X-ray imaging system 10 for acquiring original image data and processing the image data for display. The digital X-ray imaging system 10 includes an X-ray radiation source 12 positioned adjacent to a collimator 14. The collimator 14 permits a stream of X-ray radiation 16 to pass into a region in which an object or subject 18 is positioned.

A portion of the X-ray radiation 20 passes through or around the object or subject 18 and impacts a digital X-ray detector 22. In certain embodiments, the detector 22 may include a complementary metal-oxide-semiconductor (CMOS) based detector. As will be appreciated by those skilled in the art, the digital X-ray detector 22 may convert the X-ray radiation photons received on its surface to lower energy light photons, and subsequently to electric signals, which are acquired and processed to reconstruct an image of the features within the object or subject.

The X-ray radiation source 12 is controlled by a power supply/control circuit 24 which supplies both power and control signals for examination sequences. Moreover, the digital X-ray detector 22 is communicatively coupled to a detector controller 26 which commands acquisition of the signals generated in the detector 22. In certain embodiments, the detector 22 may communicate with the detector controller 26 via any suitable wireless communication standard, although the use of digital X-ray detectors 22 that communicate with the detector controller 26 through a cable, tether or some other mechanical connection are also envisaged. The detector controller 26 may also execute various signal processing and filtration functions, such as for initial adjustment of dynamic ranges, interleaving of digital image data, and so forth.

Both the power supply/control circuit 24 and the detector controller 26 are responsive to signals from a system controller 28. In general, the system controller 28 commands operation of the imaging system to execute examination protocols and to process acquired image data. In the present context, the system controller 28 also includes signal processing circuitry, typically based upon a programmed general purpose or application-specific digital computer, associated memory circuitry, such as optical memory devices, magnetic memory devices, or solid-state memory devices, for storing programs and routines executed by a processor of the computer to carry out various functionalities, as well as for storing configuration parameters and image data, interface circuits, and so forth.

In the embodiment illustrated in FIG. 1, the system controller 28 is linked to at least one output device, such as a display or printer 30. The output device may include standard or special purpose computer monitors and associated processing circuitry. One or more operator workstations 32 may be further linked in the system for selecting system parameters, requesting examinations, viewing images, and so forth. In general, displays, printers, workstations, and similar devices supplied within the system may be local to the data acquisition components, or may be remote from these components, such as elsewhere within an institution or medical facility, or in an entirely different location, linked to the image acquisition system via one or more configurable networks, such as the Internet, virtual private networks, and so forth.

The digital X-ray imaging system 10 as shown in FIG. 1 may also include a variety of alternative embodiments generally configured to meet the particular needs of certain applications. In certain embodiments, the digital X-ray imaging system 10 may be a fixed or mobile radiography system, a tomosynthesis system, a fluoroscopy system, a computed tomography (CT) system, or any combination thereof. As examples, the digital X-ray imaging system 10 may be a fixed radiography or tomosynthesis system where the digital X-ray detector 22 is either permanently mounted together with the system or portable for positioning in a table or wall stand, a mobile radiography system where the digital X-ray detector 22 is portable or tethered, a fixed fluoroscopy system where the digital X-ray detector 22 is permanently mounted within the system, or a mobile fluoroscopy system where the digital X-ray detector 22 is permanently positioned opposite of the X-ray radiation source 12.

Throughout the following discussion, while basic and background information is provided on the digital X-ray imaging system used in medical diagnostic applications, it should be born in mind that aspects of the present subject matter may be applied to digital X-ray detectors used in different settings (e.g., projection imaging, computed tomography imaging, and tomosynthesis imaging, etc.) and for different purposes (e.g., parcel, baggage, component, and part inspection, etc.).

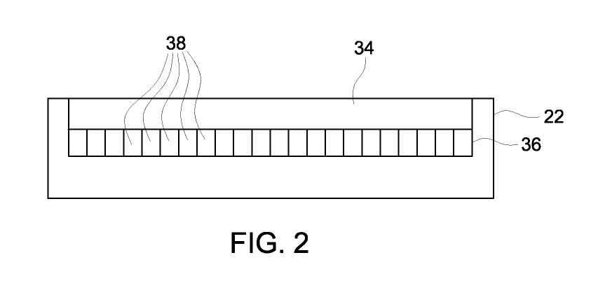

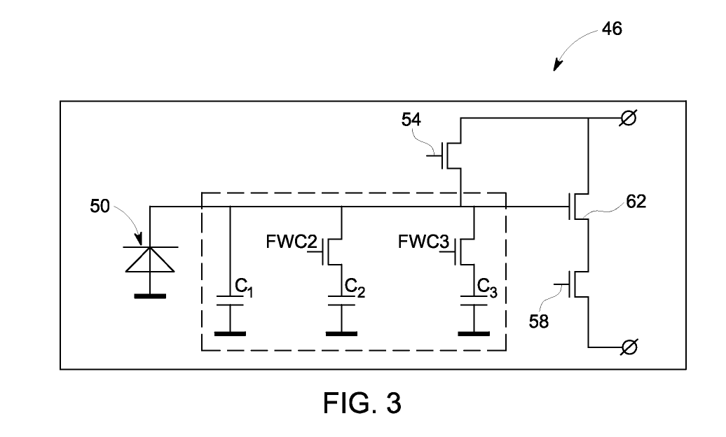

FIG. 2 illustrates a cutaway side view of a portion of the detector 22. The detector 22 includes a scintillator 34 mounted on or deposited directly onto an image device 36. The image device 36 includes a CMOS image sensor array of pixel sensors 38. By way of example only, the size of the pixel sensor array may be 1,536 by 1,536. FIG. 3 illustrates a diagram of a circuit 46 associated with each pixel sensor 38. The circuit 46 includes a photodetector or pinned photodiode 50, capacitors C1, C2, and C3, a reset gate 54, a select gate 58, and a source-follower readout transistor 62. Alternatively, the circuit 46 of the pixel 34 may be configured to include two capacitors or more than three capacitors.

To take an x-ray image with the system 10, the ROI of a patient is positioned between the x-ray source 12 and the detector 22. The controller 28 activates the reset gate 54 of the pixel 38 to connect the photodiode 50 to the power supply and remove any charge from the photodiode 50. The x-ray source 12 emits an x-ray dose at the ROI and detector 22. The scintillator 34 absorbs the x-rays that reach the detector 22 and converts the x-rays into visible light photons. The photodiodes 50 of the pixels 38 of the image device 36 convert the visible light photons into electrons and the electrons are stored in at least capacitor C1 of the pixel 38. The electrons are amplified at the source follower transistor 62 and are transferred via the select gate 58 to an analog-to-digital (A/D) converter, which converts the electrons to a digital signal that is sent via the controller 28 to the display 30 create and display an image of the ROI based on the digital signal.

When using the system 10, an operator can use the controller 28 through the operator workstation 32 to choose from and apply a number of different x-ray dose ranges. Different clinical applications may require different x-ray dose ranges. For example, an operator can choose to apply a lower dose in the range of 50 μR/frame for fluoroscopic imaging of a chest or abdomen region, a medium dose in the range of 250 μR/frame for digital cineradiography imaging, or a larger dose in the range of 500 μR/frame for digital spot imaging. Alternatively, the operator can use the workstation 32 to simply input or select an exam type, such as a fluoroscopy exam, a cineradiography exam, or a digital spot exam, and the controller 28 will then automatically select the appropriate x-ray dose range for the particular exam selected. For example, if the operator selects a fluoroscopic exam, the controller 28 will cause the x-ray source 12 to apply an x-ray dose in the range of 50 μR/frame. If the operator selects a digital cineradiography exam, the controller 28 will cause the x-ray source 12 to apply an x-ray dose in the range of 250 μR/frame. If the operator selects a digital spot exam, the controller 28 will cause the x-ray source 12 to apply an x-ray dose in the range of 500 μR/frame.

The greater the x-ray dose range, the larger the capacitor size that is needed for each pixel 38 to store the electrical charge created by the photodiode 50 of each pixel 38. However, the larger the capacitance of each pixel 38 (and thus the larger the dynamic range of the x-ray detector 22), the greater the image noise due to electronic noise. Thus, the number of capacitors C1, C2, and C3 that are operatively connected to the photodiode 50 of each pixel 38 depends on the size of the x-ray dose range for a particular clinical application. For example, when just capacitor C1 of each pixel 38 is connected the photodiode 50 of each pixel 38, the image device 36 has a dynamic range that is capable of holding the charge associated with an x-ray dose of up to 50 μR/frame that is received by the detector 22. Thus, for procedures that use an x-ray dose of up to 50 μR/frame, capacitor C1 is the only capacitor of each pixel 38 that is connected to the photodiode 50, and capacitors C2 and C3 are not connected to the photodiode 50.

However, when each pixel 38 of the image device 36 has only capacitor C1 connected to the photodiode 50, the dynamic range of the image device 36 may not be large enough to hold the entire charge associated with x-ray dose ranges that are greater than 50 μR/frame. But when both capacitors C1 and C2 of each pixel 38 are connected to the photodiode 50 of each pixel 38, the dynamic range of the image device 36 is large enough to hold the charge associated with x-ray doses in the medium range of 50-250 μR/frame. Thus, for doses of such a medium range, the controller 28 connects capacitor C2 of each pixel 38 in the image device 36 to the photodiode 50 of the pixel 38 by activating gate FWC2 of the circuit 46, which results in each pixel 38 of the image device 36 having both capacitor C1 and capacitor C2 operatively connected to the photodiode 50. Similarly, for x-ray doses in a range that is greater than 250 μR/frame, the dynamic range of the image device 36 may not be great enough to hold the charge associated with the dose if, for each pixel 38 in the image device 36, only capacitors C1 and C2 are connected to the photodiode 50. Therefore, for such larger x-ray doses, the controller 28 connects both capacitors C2 and C3 of each pixel 38 to the photodiode 50 of the pixel 38 by activating gates FWC2 and FWC3, respectively, of the circuit 46.

Alternatively, each pixel 38 in the image device 36 is not limited to having exactly three capacitors. Depending on the desired capacitance of each capacitor connected to the photodiode 50, the desired total possible capacitance for each pixel 38, and the number of pixels 38 in the image device 36, each pixel 38 may have only two capacitors that can be independently connected to the photodiode 50 or may have more than three capacitors that can be independently connected to the photodiode 50.

Therefore, depending on the x-ray dose range for a procedure, a different dynamic range for the image device 36 can be selected by connecting a different number of capacitors to the photodiode 50 of each pixel 38. The greater the x-ray dose range, the greater the number of capacitors that can be operatively connected to the photodiode 50 of each pixel 38 to accommodate the charge received from the photodiode 50 of the pixel 38. In this way, the amount of electronic noise created by the size of the charge the image device 36 can hold, i.e., the dynamic range of the image device 36, can be reduced for procedures that require a smaller x-ray dose range. Thus, instead of each pixel 38 having one large capacitor (and thus the image device 36 having one single large dynamic range) that can accommodate all x-ray dose ranges and that creates the same large amount of electronic noise regardless of the size of the x-ray dose range, the capacitance of each pixel 38 can be reduced (and thus the dynamic range of the image device 36 can be reduced) for smaller x-ray doses in order to reduce the electronic noise created by excess capacitor size.

Moreover, since quantum noise increases with the size of the x-ray dose, for those clinical applications that require a large x-ray dose, and thus a greater dynamic range for the detector 22, the increase in electronic noise that comes with a greater dynamic range is still small compared to the increase in quantum noise. For example, where the dose range is increased from 50 μR/frame to 250 μR/frame, the increase in electronic noise due to the increase in dynamic range of the image device 36 (i.e., due to the capacitance of each pixel 38 being increased from the capacitance of capacitor C1 to the capacitance of capacitors C1 and C2), may be no more than 0.5% of the increase in quantum noise due to the increase of the dose range.

Thus, the system 10 provides multiple dynamic range selections that can be used to reduce image noise by limiting the electronic noise associated with x-ray procedures using particular x-ray dose ranges. In particular, for smaller x-ray doses, the system 10 can use smaller dynamic ranges by reducing or limiting the size of the capacitance for each pixel 38 in the detector 22. By using smaller dynamic ranges for smaller x-ray doses, the contribution of electronic noise to image noise is less than that of a system that only includes only one large dynamic range to accommodate all x-ray dose sizes. Moreover, even for the larger dynamic ranges of the system 10, which are used to accommodate larger x-ray doses, the contribution of the electronic noise to the total image noise is still low compared to the contribution of the quantum noise associated with the x-ray dose size to the total image noise.

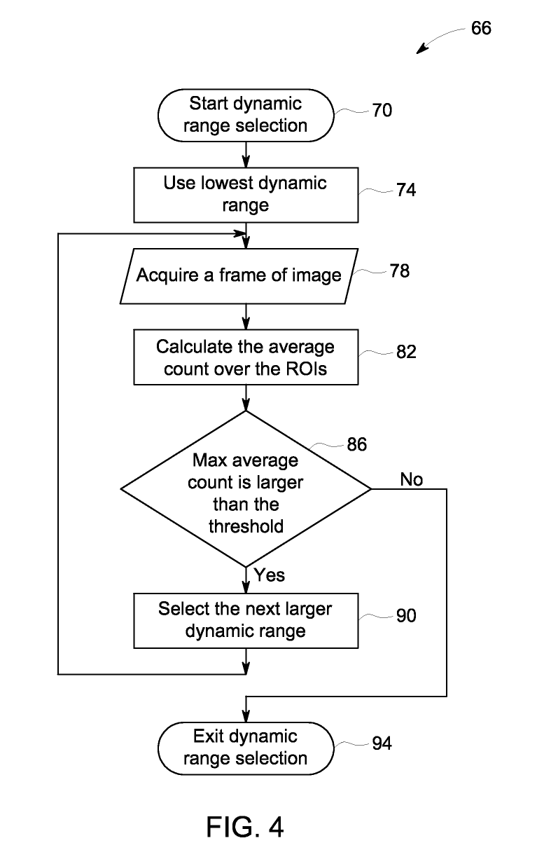

Moreover, the controller 28 of the x-ray system 10 can operate in a dynamic range selection mode that automatically selects an appropriate dynamic range for a particular x-ray procedure based on the count level of electrons received by the detector 22 for the procedure. FIG. 4 illustrates a flow chart for the dynamic range selection mode 66. As shown in the flow chart, the first step 70 is that an operator uses the operator workstation 32 to start the dynamic range selection mode. The controller 28 selects the lowest dynamic range of the system 10 (step 74), i.e., for each pixel 38, only capacitor C1 is connected to the photodiode 50. An appropriate x-ray dose for the particular x-ray procedure is then applied to the ROI, and a frame of an image of the ROI is acquired from the image device 36 of the detector 22 (step 78). The controller 28 calculates the maximum average count level of electrons received by the detector 22 among smaller regions of interest located within the entire ROI (step 82). This maximum average count level is compared to a pre-selected threshold count level (step 86). For example, the threshold count level is the maximum number of electrons or charge that can be held by the image device 36 when the image device 36 is set at the lowest dynamic range, i.e., for each pixel 38 in the image device 36, only capacitor C1 is connected to the photodiode 50.

If that maximum average count is larger than the threshold count level, the controller 28 selects the next lowest dynamic range of the system 10 (step 90), i.e., for each pixel 38, the controller 28 connects capacitor C2 to the photodiode 50 so that the total capacitance of each pixel 38 is the combined capacitance of capacitors C1 and C2. The system 10 then acquires another frame of an image and the average count for the image is again calculated (steps 78 and 82). That count is then compared to a new threshold count level that relates to the maximum number of electrons that can be held by the image device 36 when, for each pixel 38 in the image device 36, capacitors C1 and C2 are connected to the photodiode 50 (step 86). If the maximum average count is larger than the new threshold count level, the process repeats itself and the controller 28 selects the next lowest dynamic range (step 90), i.e., for each pixel 38, the controller 28 connects capacitor C3 to the photodiode 50 so that the total capacitance of each pixel 38 is the combined capacitance of capacitors C1, C2, and C3, and selects a new threshold count level for the new larger dynamic range.

However, once the maximum average count is determined to be no larger than the threshold count level for a dynamic range, the system 10 exits the dynamic range selection mode 66 (step 94), and the system 10 is set at the appropriate dynamic range for the particular x-ray dose being used. Thus, the dynamic range selection mode selects the smallest dynamic range that is still large enough to hold the electronic charge associated with the x-ray dose of a particular procedure. In this way, the dynamic range selection mode 66 serves to limit the contribution of electronic noise to the overall image noise associated with a particular imaging procedure by limiting the size of the dynamic range of the system 10 for the procedure.

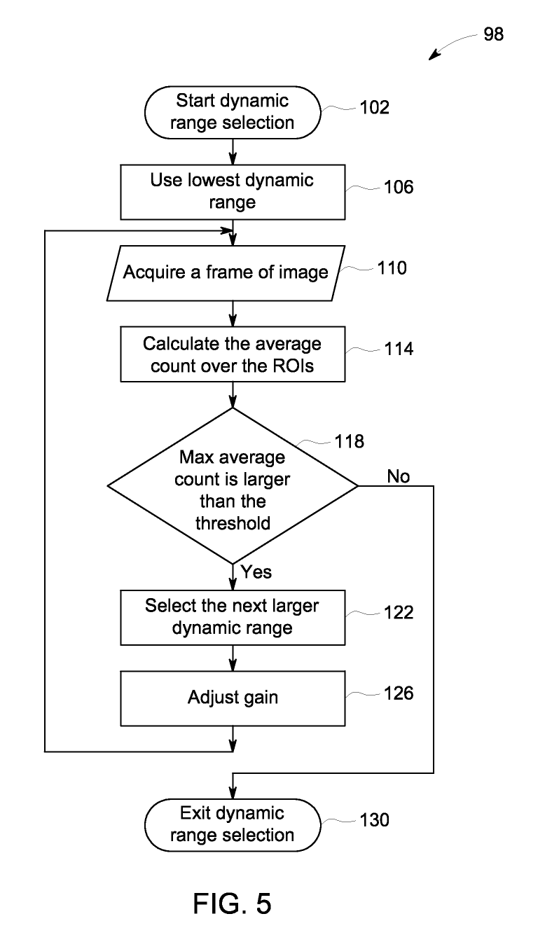

In addition, in another embodiment, the controller 28of the x-ray system 10 operates in a dynamic range selection mode similar to that discussed above but that also includes an automatic brightness mode. The process for this dynamic range selection mode 98 with automatic brightness adjustment is shown in the flow chart at FIG. 5. As with the mode 66 shown in the flow chart at FIG. 4, when the mode 98 of FIG. 5 is selected (step 102), the controller 28 selects the lowest dynamic range of the system 10, (step 106), an x-ray dose for a particular procedure is applied to the ROI, and a frame of the image of the ROI is acquired (step 110). The controller 28 calculates the maximum average count level of electrons received by the detector 22 among smaller regions of interest located within the entire ROI (step 114) and the maximum average count level is compared to a threshold count level for the lowest dynamic range (step 118). If that maximum average count is larger than the threshold count level, the next lowest dynamic range is selected (step 122).

After the next lowest dynamic range is selected, the automatic brightness compares the gain, i.e., the ratio of electrons stored in the capacitors to the digital count upon analog to digital conversion, when the detector 22 is using the lowest dynamic range to the gain for the detector 22 when the detector 22 is using the next lowest dynamic range. The controller 28 adjusts for the change in the gain between the two different dynamic ranges such that the digital count for an image created and displayed using the next lowest dynamic range is the same as the digital count for the image created using the lowest dynamic range (step 126). The system 10 then acquires an image using the next lowest dynamic range and the process is repeated (steps 114 and 118) until the maximum average count for an image is not greater than the threshold count level for the dynamic range being used. At this point, the system 10 exits the dynamic range selection and automatic brightness mode 98 (step 130) and the appropriate dynamic range has been selected for the particular x-ray procedure. By adjusting for changes in the gain due to changing from one dynamic range to another dynamic range, the digital count associated with each image is similar and thus images taken using different dynamic ranges are similar in brightness and appearance.

Embodiments of the present invention provide a system and method for selecting a dynamic range for a particular x-ray imaging procedure that limits the electronic noise that is created as a result of the procedure. In particular, embodiments of the present invention provide for the selection of the number of capacitors that are connected to a photodiode for each pixel in an x-ray imaging system so as to limit the total capacitance, and thus the dynamic range, of the system for an x-ray procedure and thus limit the contribution of electronic noise to the noise of an image acquired from the procedure. Embodiments of the present invention also provide for a dynamic range selection mode that adjusts the dynamic range for a procedure based on the average electron count of an acquired image. Embodiments of the present invention also provide for a dynamic range selection mode that adjusts the dynamic range for a procedure based on the average electron count of an acquired image and the brightness of an image based the change in gain due to using different dynamic ranges.

While various spatial and directional terms, such as top, bottom, lower, mid, lateral, horizontal, vertical, front and the like may used to describe embodiments of the present invention, it is understood that such terms are merely used with respect to the orientations shown in the drawings. The orientations may be inverted, rotated, or otherwise changed, such that an upper portion is a lower portion, and vice versa, horizontal becomes vertical, and the like.

Variations and modifications of the foregoing are within the scope of the present invention. It is understood that the invention disclosed and defined herein extends to all alternative combinations of two or more of the individual features mentioned or evident from the text and/or drawings. All of these different combinations constitute various alternative aspects of the present invention. The embodiments described herein explain the best modes known for practicing the invention and will enable others skilled in the art to utilize the invention. The claims are to be construed to include alternative embodiments to the extent permitted by the prior art.

Various features of the invention are set forth in the following claims.

1. An x-ray system, comprising:
an x-ray source; and
an x-ray detector, said x-ray detector including a scintillator and at least one pixel, wherein said pixel includes:
a photodiode;
a first capacitor connectable to said photodiode; and
a second capacitor connectable to said photodiode, said at least one pixel being configured to be switched between a first state where only said first capacitor is operatively connected to said photodiode and a second state wherein said first and second capacitors are both operatively connected to said photodiode;
wherein said x-ray source directs x-rays at a region of interest positioned between said x-ray source and said x-ray detector, said scintillator converts the x-rays to light, and said photodiode of said at least one pixel converts the light to an electrical charge, said charge being stored in the first capacitor when said at least one pixel is in said first state and said charge being stored in said first and second capacitors when said at least one pixel is in said second state.

2. The x-ray system of claim 1, wherein said at least one pixel includes a third capacitor connectable to said photodiode.

3. The x-ray system of claim 2, wherein said at least one pixel is configured to be switched from said first or second state to a third state wherein said first, second, and third capacitors are all operatively connected to said photodiode.

4. The x-ray system of claim 1, further including a controller that converts the electrical charge to a digital signal and creates an image of the region of interest from the digital signal.

5. The x-ray system of claim 1, wherein said x-ray detector includes an array of pixels.

6. The x-ray system of claim 1, wherein when said at least one pixel is in said second state, the capacitance of said at least one pixel is greater than the capacitance of said at least one pixel when said at least one pixel is in said first state.

7. The x-ray system of claim 1, wherein said detector is a complementary metal oxide semi-conductor detector.

8. The x-ray system of claim 1, further comprising a controller that switches said at least one pixel from said first state to said second state when the average count of electrons generated by said photodiode upon converting light to a charge is greater than a threshold that corresponds to the number of electrons that can be stored by said x-ray detector when said at least one pixel is in said first state.

9. A method for changing the maximum electrical charge that an x-ray detector can hold, comprising:
providing an x-ray system having an x-ray source and an x-ray detector connected to a controller, wherein said x-ray detector includes a scintillator and at least one pixel including a photodiode connectable to a first capacitor and a second capacitor;
selecting, with said controller, to connect only the first capacitor to the photodiode or to connect both the first capacitor and the second capacitor to the photodiode;
directing an x-ray dose from the x-ray source at a region of interest positioned between the x-ray source and x-ray detector;
converting the x-rays of the x-ray dose to light with the scintillator;
converting the light into an electrical charge with the photodiode of the at least one pixel and storing the charge in the first capacitor if only the first capacitor was selected to be connected to the photodiode or in the first and second capacitors if both the first and second capacitors were selected to be connected to the photodiode;
converting the charge into a digital signal; and
converting the digital signal to an image of the region of interest.

10. The method of claim 9, further comprising providing a third capacitor that is connectable to the photodiode of the at least one pixel.

11. The method of claim 10, wherein said selecting step comprises selecting with said controller to connect only the first capacitor to the photodiode, or to connect only the first and second capacitors to the photodiode, or to connect the first, second, and third capacitors to the photodiode.

12. The method of claim 9, further comprising selecting with the controller a particular type of x-ray procedure.

13. The method of claim 12, wherein said controller selects to connect only the first capacitor to the photodiode or to connect both the first capacitor and the second capacitor to the photodiode based on the type of x-ray procedure selected.

14. The method of claim 13, wherein the x-ray procedures that can be selected include at least one of a fluoroscopy procedure, a cineradiography procedure, and a digital spot procedure.

15. A method for changing the maximum electrical charge that an x-ray detector can hold, comprising:
providing an x-ray system configured to create an x-ray image of a region of interest and having an x-ray source and an x-ray detector connected to a controller, wherein said x-ray detector includes at least one pixel including a photodiode connectable to a first capacitor and at least a second capacitor;
connecting, with the controller, only the first capacitor to the photodiode and leaving the at least a second capacitor unconnected to the photodiode;
positioning a region of interest between the x-ray source and x-ray detector and directing x-rays at the region of interest to acquire an image of the region of interest;
calculating, with the controller, the average count of electrons received by the detector from the step of directing x-rays at the region of interest; and
comparing, with the controller, the average count of electrons to a threshold count of electrons that corresponds to the number of electrons that can be held by the at least one pixel when the photodiode of the at least one pixel is connected to the first capacitor.

16. The method of claim 15, further including selecting, with the controller, to connect the at least a second capacitor to the photodiode of the pixel if the average count is greater than the threshold count.

17. The method of claim 16, further including changing the threshold count to a second threshold count that correspond to the number of electrons that can be held by the at least one pixel when the photodiode of the at least one pixel is connected to the first capacitor and the at least a second capacitor.

18. The method of claim 17, further including positioning a region of interest between the x-ray source and x-ray detector, directing x-rays at the region of interest to acquire an image of the region of interest, calculating, with the controller, the average count of electrons received by the detector from the step of directing x-rays at the region of interest, and comparing, with the controller, the average count of electrons to the second threshold count of electrons that corresponds to the charge that can be held by the first capacitor and the at least a second capacitor.

19. The method of claim 19, further including selecting, with the controller, to connect a third capacitor to the photodiode of the pixel if the average count is greater than the second threshold count.

20. The method of claim 16, further including adjusting the gain associated with a second image taken with the first and at least second capacitor connected to the photodiode in order to adjust the brightness of the second image.
